(19) United States
(12) Patent Application Publication
(10) Pub. No.: US 2022/0241552 A1
(43) Pub. Date: Aug. 4, 2022

(54) ACTIVELY DEFLECTABLE URINARY CATHETER

(71) Applicant: Middleton Medical Innovations Pty Ltd, Camberwell, Victoria (AU)

(72) Inventors: Ian S. Middleton, Lenah Valley (AU); Dougal L. Middleton, Cairns, (AU); Rory L. Middleton, South Melbourne (AU)

(21) Appl. No.: 17/595,043

(22) PCT Filed: Nov. 16, 2020
(86) PCT No.: PCT/AU2020/051240

§ 371 Date: Nov. 5, 2021

(30) Foreign Application Priority Data
Nov. 20, 2019 (AU) 2019904394

(51) Int. Cl. A61M 25/00

(52) U.S. Cl. CPC A61M 25/008; A61M 25/0026; A61M 2210/1089; A61M 2025/0037; A61M 2025/0081

(57) ABSTRACT

A urinary catheter with an actively deflectable tip, the tip portion being deflectable for the purpose of steering the catheter within the urethra to allow the catheter to follow a natural curvature of the urethra, when inserted into the urethra by a user. The described catheter includes features of the catheter described in PCT/AU2020/050972, but includes a number of modifications and variations, which may allow additional benefits to those of the previously described catheter.

The present invention relates to a urinary catheter and method of catheterising the bladder of a male patient. This application represents an extension of international patent application no. PCT/AU2020/050972 and contains variations and modifications of the catheter described in that application.

### FIELD OF THE INVENTION

### Background

Inserting a bladder catheter into the urethra of a male patient may be required in many medical situations. It is a common task for hospital medical and nursing staff, and it can be a difficult procedure due to the curvature of the male urethra as it passes up through the prostate gland, particularly if there has been previous prostatic surgery. If difficulty is encountered, a urological surgeon is often called to assist.

At present, three common options used for catheterisation of a male urethra include a simple straight flexible urethral catheter, a Coude tip catheter with a fixed curve and a simple straight catheter into which a rigid catheter introducer is inserted.

A simple straight flexible urethral catheter is lubricated and simply pushed up the urethra, and in the majority of cases, will follow the natural curve of the urethra and enter the bladder without significant trauma to the urethra. Generally straight urinary catheters have limited flexibility, because the catheter has to be stiff enough to push up the urethra. This limited flexibility may not be sufficient to allow the catheter to follow the urethral curve unless the urethra is straightened by the catheter, and this may result in urethral damage or a so called ‘false passage’ in the wall of the urethra.

Also, if there is a slight irregularity of the urethral lining, or there is scarring or more major deviations in the urethral tube, the catheter will simply not pass, and in a significant number of cases, the catheter tip causes at least some damage to the urethra. Studies have shown that catheterisation can be difficult in up to 20% of cases. Presently available generally straight catheters cannot be steered during the catheterisation process.

In an attempt to overcome this problem, catheters with a fixed curved tip (Coude tip) have been devised and produced, to facilitate the passage of the catheter around the curved urethra. These catheters however are not truly steerable and will only pass readily if the curve of the particular urethra happens to be the same as that of the catheter tip. The fixed curve of the catheter tip also often makes passage of the catheter difficult through the distal part of the urethra, which is straight. In practice, the curve chosen during the design of a Coude tip catheter is a compromise between the full angle of the curved urethra and the zero degree angle of the straight section of the urethra.

If a catheter introducer is used, this curved, rigid metal rod is inserted into a simple, straight, flexible catheter prior to passage of the catheter up the urethra. Because it is curved to match the full curve of a male urethra, it is difficult and potentially dangerous to pass up the straight section of the urethra but is shaped to have enough angle of curvature to comply with the full curve of the urethra. Because catheter introducers are rigid and will cause major trauma to the urethra if not perfectly inserted, they are usually not used by any medical professionals apart from actual urologists.

The problems noted above have resulted in a need for a new catheter design which will have all or most of the advantages of both a Coude tip catheter and an introducer, but few or preferably none of the disadvantages. What is needed is a truly steerable urethral catheter, which is straight during passage of the straight distal urethra and bent during passage of the curved proximal urethra.

Steerable devices including telescopes and catheters have been developed to navigate within a number of body organs including the bowel and blood vessels, (eg GB1116317, CN1088536689 and CN203183482)), and also within parts of the urinary tract, including the bladder and kidneys. These devices utilise various types of steering mechanism, which may include steering wires and a steering control mechanism. Guidance may be by direct visualisation via a telescope (eg WO2014043586) or camera, or by radiological guidance utilising various modalities including fluoroscopy (eg WO2019152727). Each device may have individual characteristics which make it suitable for that particular body location, and the exact purpose for which it was intended, but may make it completely unsuitable for use as a urinary catheter steerable within the urethra itself.

As an example, the cystoscopy catheter documented in WO2014043586 is designed to act as both a flexible, potentially steerable, sheathe for a cystoscope or therapeutic instrument within the bladder, and an indwelling bladder catheter, but it is not designed to be deflected within the urethra or to facilitate passage of the catheter up the urethra.

As another example, the catheter in WO2019152727, is designed to be deflectable only within the kidney, and is not designed to be deflected within the urethra or to facilitate passage of the catheter up the urethra or to drain the urinary bladder.

As noted above, a significant problem for the management of male patients who require a urinary catheter is the actual passage of the catheter up the urethra and into the bladder, because of the curved and irregular shape of the male urethra. Previous catheter designs have not allowed adequate manipulation of the catheter within the urethra to accommodate the individual anatomy of the particular urethra to be catheterised. What is required is a steerable catheter with particular design features which will make it suitable for that purpose. These characteristics are different in many respects to devices which may have been designed to be steerable in other parts of the urinary tract.

Because bladder catheterisation via the urethra has to be performed in the community, in patients' homes, in doctors' offices, in emergency departments and on hospital wards, it must be possible to perform catheterisation in the majority of cases without the need for telescopic or radiological guidance, which are too expensive and impractical to use routinely for what is generally a minor procedure. As such, the catheter must be insertable by tactile sensation or ‘feel’, as described further below.

The procedure must also be suitable to be performed by community and hospital nurses, other health care workers, junior doctors and indeed the patients themselves in certain circumstances (self-catheterisation), and a complex steering mechanism is completely unsuitable for such purpose.

The appropriate catheter should be designed specifically to be steerable within the urethra, which does not require greate deflection, or rotation, as is necessary within the bladder or kidney. This is because of the specific anatomical characteristics of the urethra compared to other parts of the urinary tract. The design also needs to take into account the anatomical reasons why the catheter may be impeded, as described in more detail later.

The angle of curvature of a catheter designed to be steered up the male urethra is completely different to the angle required for a catheter or telescope designed to function optimally in the bladder or kidney. In particular, curvature of the urethra is usually more than 45 degrees upwards, and never more than 90 degrees. In contrast, the angle of curvature of a catheter in the kidney needs to be acute, ideally to over 90 degrees in opposite directions, and with a much smaller radius of arc, if it is to fit within the tight confines of the kidney collecting system. To obtain the necessary arc of deflection, the deflecting segment of the tip needs to be significantly longer, of the order of 5 to 6 cms. A deflectable device in the bladder also needs to be able to deflect to more than 90 degrees if it is to be able to access all surfaces of the bladder, including the bladder neck, and the arc of deflection would normally be much smaller than the gradual deflection which is optimal for the urethra. Such acute deflection of a catheter in the urethra would just result in impingement of the tip of the catheter on the anterior urethral wall.

As the proximal urethra does not deviate laterally but passes in the same sagittal plane throughout its curved course, the catheter only needs to be able to deflect upwards from the horizontal and does not have to be able to deflect laterally or be rotated. Because the only variation in a normal urethra is therefore in its length, and the actual angle of the upward curvature, the ideal actively deflectable catheter only has to be able to be deflectable between zero and 90 degrees to the horizontal, and in the same sagittal plane. The natural elastic recoil of the catheter will result in straightening of the catheter tip if traction is decreased or removed.

A fully steerable device is therefore not required and would necessitate a bigger, heavier and more complex steering mechanism, such as may be required in the bladder or kidneys. Such a complex mechanism is likely to be expensive to produce, and also less likely to be suitable to be disposable, which is a virtual pre-requisite for a urethral catheter.

The anatomy of the male urethra is illustrated in detail in PCT/AU2020/050972, as well as the problem associated with the use of currently available urethral catheters.

The problems shown demonstrated that there is a need for a new type of catheter suitable for inserting into the bladder via the urethra. The deflectable tip of the catheter must be configured to the specific anatomy of the male urethra and the steering mechanism designed for the requirements of a disposable device.

The deflection mechanism preferably should be simple, cost effective, very light, easy and cheap to manufacture, not affected by contact with fluids, easily sterilisable with the catheter, preferably without metallic components (so that removal is not required for patients needing an MRI scan), able to be readily operated with a single finger even by a disabled person, and/or at least provide a useful alternative to what is available at present.

That application describes a urinary catheter with a flexible, actively deflectable catheter tip, utilizing a synthetic steering member. The specification describes several mechanisms which can be built into the catheter, to enable the person introducing the catheter to deflect the tip upwards, to help it follow the natural curvature of the male urethra.

The described deflection mechanisms allow active deflection upwards of the catheter tip but depend on the inherent elasticity and resilience of the catheter tube to passively return the curved tip of the catheter to the undeflected state, once manual tension on the steering member is released. At least a degree of straightening of the catheter tip after deflection may aid insertion of the catheter and provide additional controllability of the catheter tip to allow precise navigation of the male urethra.

A variation in the deflection mechanism is now described which allows active deflection of the catheter tip either upwards or downwards. As well as providing the possibility of greater control of the catheter tip, not having to rely on catheter elasticity may allow the catheter to be manufactured softer and more easily deflected by the urethral lining tissue, and therefore make the catheter tip less likely to cause trauma to the urethra.

The catheter tip described in application PCT/AU2020/050972 is tapered to a thin walled distal section, and because the wall of the tip is thinner than the rest of the catheter, it will readily deflect with traction on the steering element, whereas the thicker and stiffer walled proximal catheter will remain virtually undeflected.

In another embodiment of the catheter, the tip portion is manufactured such that the distal tip portion is comprised of material which is more flexible than that material in the elongate portion of the catheter. Such a configuration would allow for different deflectability of the distal tip portion, without the need for different catheter wall thickness along its length. (See FIG. 4).

Because the catheter tip described in PCT/AU2020/050972 is generally cylindrical in shape, there may be a tendency for lateral torsion to occur when the tip is deflected upwards, and therefore one embodiment of the invention described in that application utilizes two parallel steering elements dorsally, designed to minimize lateral torsion and to facilitate direct upward rather than lateral deflection.

A number of variations in the shape of the catheter tip section are described whereby the tip may be manufactured in a number of configurations. As an example, in one embodiment of the catheter, the tip is a flattened oval shape rather than a cylinder with a circular cross-section. The flattened cylinder is of a lesser diameter in the superior to inferior plane, as opposed to the lateral plane, and in another variation, there can be a moderately sharp angulation at the lateral edges of the catheter. It is envisaged that these and similar variations in the catheter tip should facilitate the tip section to deflect directly up or down rather than laterally.

Resistance to lateral deflection can also be facilitated by the lateral wall thickness of the flattened cylinder being greater than that of the wall thickness of the superior and inferior walls of the catheter tip.

These variations in catheter tip shape may also potentially be utilized to aid the insertion of an otherwise conventional Foley catheter, without any deflecting elements. Several examples are shown in FIGS. 10a and 10b.

In another embodiment, the catheter tip can also be manufactured with a slight upwards angulation, to help facilitate passage of the catheter through the upward curving urethra. As with the flattened, oval shaped catheter tip described in the last paragraph, this angulated catheter tip has potential advantages in any catheter, even conventional catheters without a deflecting mechanism. Unlike a Coude tip catheter however, the tip of the new catheter is angulated up minimally so that the tip does not protrude outside the line of the shaft of the proximal catheter tube and is thus less likely to traumatize the urethral lining tissue on passage of the catheter.

Overall, the described variations in the original specification of the catheters described in international patent application PCT/AU2020/050972 have the potential to enhance further the advantages of the invention.

### SUMMARY OF INVENTION

According to one aspect of the invention there is provided a urinary catheter, comprising:

a hollow catheter body configured to be inserted within the urethra of a patient, the catheter body having an elongate portion, a tip portion near the distal end of the elongate portion and an engagement portion spaced from the distal end where a user can grasp the catheter, the tip portion being deflectable for steering the catheter within the urethra to allow the catheter to follow a natural curvature of the urethra, the tip portion movable from a generally straight condition to a deflected condition wherein deflection of the tip portion occurs while the elongate portion remains generally straight,

wherein the catheter body has a main channel formed therein for draining urine from a bladder and at least two further channels formed remote from the main channel and in which flexible steering members can be situated, with each further channel extending from the distal end to a location near the engagement portion with the steering members being secured within the further channels at the tip portion and configured for engagement by a user externally of the catheter to deflect the tip portion.
a. According to preferred embodiments of the invention, the hollow catheter body may narrow at the tip portion.
b. Preferably, a wall thickness of the hollow catheter body reduces at the tip portion. Preferably, the tip portion is configured to deflect about an arc commencing at the intersection of the elongate portion and tip portion, the tip portion being generally straight at a distal end thereof.
c. Preferably, the elongate portion is stiffer than the tip portion and configured to remain in a generally straight condition during use. Preferably, the length of the tip portion is approximately 2 to 4 cms, although may be more or less than 2 to 4 cms. Preferably, the tip portion is configured to deflect in a plane coincident with a longitudinal axis of the elongate portion. Preferably, the tip portion can deflect through an angle up to 90 degrees.
d. Preferably, the catheter comprises further channels situated dorsally and ventrally within the catheter wall, each with a steering member received therein, which are able to deflect the tip portion of the catheter in an upwards or downwards direction.
e. The catheter may further comprise an inflatable balloon spaced from the flexible tip and a balloon inflation channel extending between the balloon and a connector at the proximal end of the catheter for communicating fluid to the balloon for inflation.
f. Preferably, the steering members are nylon or similar synthetic polymer strings. Preferably, the catheter further comprises an engagement member coupled to the ends of the steering members for engagement by a user to deflect the tip portion.
g. Preferably, the engagement member is biased into an upright position to facilitate operation by a digit of the user. More preferably, the engagement member is a lever coupled to the exterior of the catheter body at a point about which it is pivotable.
h. According to another aspect of the invention, there is provided a method of catheterising a bladder of a male patient, including the steps of:
   i. providing a catheter of the above described type;
   ii. inserting the catheter into the urethra of the patient; and
   iii. deflecting the tip portion to steer the catheter through the urethra as the catheter is inserted into the urethra.
i. The method may further include the step of retracting the catheter slightly upon encountering resistance during insertion, and then deflecting the tip portion as the catheter is re-advanced into the urethra.

### BRIEF DESCRIPTION OF THE DRAWINGS

In order that the invention may be more easily understood, embodiments will now be described, by way of examples only, with reference to the accompanying drawings, in which:

FIG. 1 is a close view of the engagement portion of a urinary catheter of one embodiment of the invention, in the undeflected state;

FIG. 2 is a close view of the engagement portion of a urinary catheter of one embodiment of the invention, which includes an engagement member limiter, in the deflected state;

FIG. 3 is a close view of the engagement portion of a urinary catheter of one embodiment of the invention, in which the engagement member can be retracted back past a vertical orientation, in the deflected state;

FIG. 4 is a sectional view of the catheter in FIG. 1, the section taken along line A-A in FIG. 1;

FIG. 5 is a sectional view of the catheter in FIG. 2, the section taken along line D-D in FIG. 2;

FIG. 6 is a sectional view of the catheter in FIG. 7, the section taken along the line C-C in FIG. 7;

FIG. 7 is a close view of the tip portion of a urinary catheter of one embodiment of the invention;

FIG. 8 is a sectional view of the catheter in FIG. 7, the section taken along the line B-B in FIG. 7;

FIG. 9a is a side view of a urinary catheter of one embodiment of the invention, in the undeflected state;

FIG. 9b is a side view of the urinary catheter of FIG. 9a, in the deflected state of use;

FIG. 10a is a sectional view of the tip portion of a urinary catheter of one embodiment of the invention, in which there are no steering members;

FIG. 10b is a sectional view of the tip portion of a urinary catheter of another embodiment of the invention, in which there are no steering members, and the wall thickness of the tip portion of the catheter is asymmetrical;

FIG. 11 is a close view of the tip portion of a urinary catheter of one embodiment of the invention, in which the tip portion is bent slightly upwards in the undeflected state;

FIG. 12a is a sectional view of the tip portion of the urinary catheter in FIG. 11, the section taken along the line E-E in FIG. 11;

FIG. 12b is a sectional view of the urinary catheter in FIG. 11, the section taken along the line F-F in FIG. 11.

FIG. 13 is a close view of the tip portion of a urinary catheter of one embodiment of the invention, in which the wall thickness of the distal tip portion is less than the wall thickness of the elongate portion of the catheter;

FIG. 14 is a close view of the tip portion of a urinary catheter of one embodiment of the invention, in which the composition of the wall of the catheter in the distal tip portion is different to the composition of the wall of the elongate portion of the catheter;

FIG. 15 is a 3 dimensional view of the engagement portion of a urinary catheter of one embodiment of the invention, in which the wall of the catheter is moulded to accommodate the engagement member and its range of movement.

### DETAILED DESCRIPTION

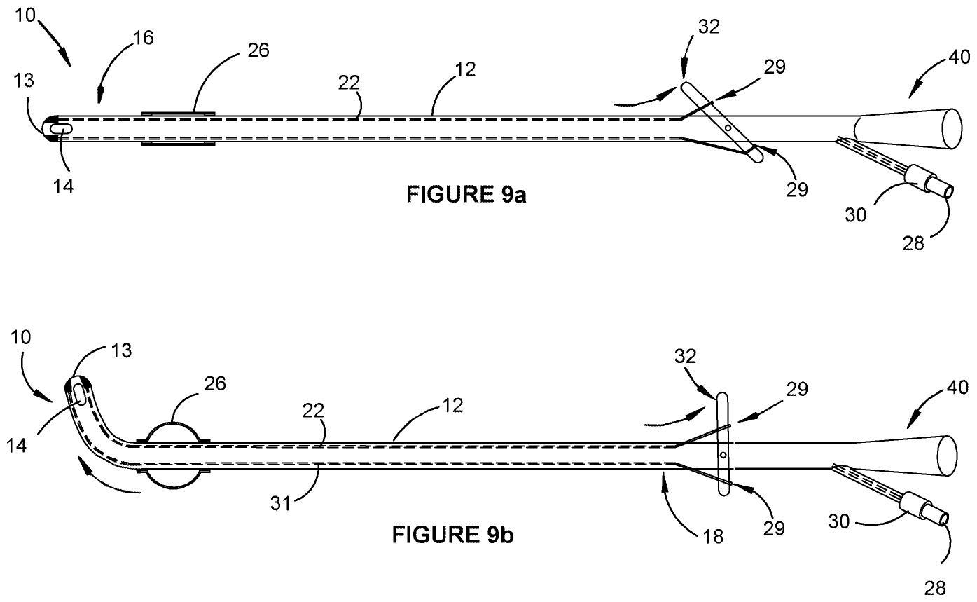

A urinary catheter 10 according to a preferred embodiment of the invention is shown in FIGS. 9a and 9b.

The urinary catheter 10 includes a hollow catheter body 12. The hollow catheter body 12 is configured to be inserted into the urethra of a patient. The catheter body 12 is elongate and has a tip portion 16 near a distal end 13 thereof and an engagement portion 18 spaced from the distal end 13 and where a user can grasp the catheter. The tip portion 16 is deflectable for steering the catheter 10 within the urethra.

The hollow catheter body 12 has a drainage aperture 14 at the distal end 13 of the catheter body 12 and through which urine can be drained from the bladder.

The engagement portion 18 of the catheter body may include a handle or grip formed on the catheter body 12 (not shown). In other embodiments, the catheter body 12 may be contoured, for example with indentations where fingers can be received, so that the engagement portion 18 is formed in the catheter body 12. In other embodiments, the catheter body may be moulded to accommodate the engagement member and its range of movement. This moulding may incorporate a hole, channel, slit or groove, through which the steering member may pass. The moulding may also be shaped so as to control or limit the range of movement of the engagement member. An example of one embodiment of such a moulding is shown in FIG. 15.

The catheter body is preferably formed of conventional materials, i.e. a silicone-based plastic using conventional moulding/forming techniques, and as a single piece. Those skilled in the art will appreciate that other materials may be used, such as latex rubber for example.

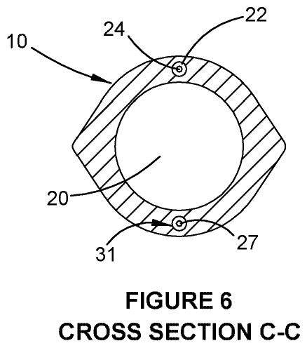

With reference to FIGS. 6 to 8, it can be seen that the catheter body 12 has a main channel 20 for draining urine from the bladder and 2 further channels 22 and 31 formed remote from the main channel 20 and in which flexible steering members 24 and 27 can be received.

Channels 22 and 31 extend from the distal end 13 to a location near the engagement portion 18 with the steering members 24 and 27 being secured within channels 22 and 31 at the distal end 13 by plugs 15 and, at an opposite end thereof, configured for engagement by a user externally of the catheter 10 to deflect the tip portion 16. This allows the catheter 10 to be steered after it is inserted into a patient's urethra. Engagement of the steering members 24 and 31 at the end remote from the distal end 13 is via the engagement member 32, which will be described in further detail below.

The hollow catheter body 12 is configured to enable deflection at the tip portion 16. This allows the catheter 10 to simulate the angle of curvature of the urethra to facilitate insertion without the above described issues. The tip portion 16 is actively deflectable by a user, i.e. under deliberate action, to facilitate insertion of the catheter 10 into the urethra.

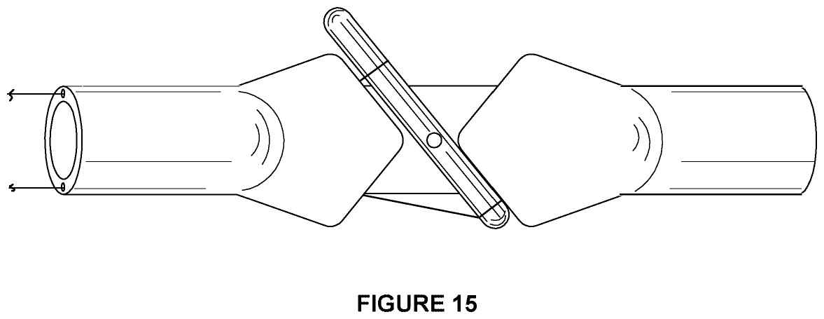

To achieve this, in some embodiments the tip portion 16 is manufactured to be more flexible than the catheter body but still have some resilience. As an example, some or all of the deflectable tip portion of the catheter may be formed of a material that is different to the material used to form the elongate portion of the catheter, as is shown in FIG. 14. If the material used to form the tip portion is more easily deflectable than the material used to form the elongate portion of the catheter, then traction on the steering element will result in preferential deflection of the tip portion, whilst the elongate portion will remain undeflected. Such a configuration would remove the necessity for the tip portion to have a thinner wall than the elongate portion of the catheter, for differential deflection to occur. After a user has removed force from the engagement member 32 that was applied to deflect the tip portion 16, the tip portion 16 straightens, although it may not return to a fully straight state due to the natural curvature of the patient's urethra.

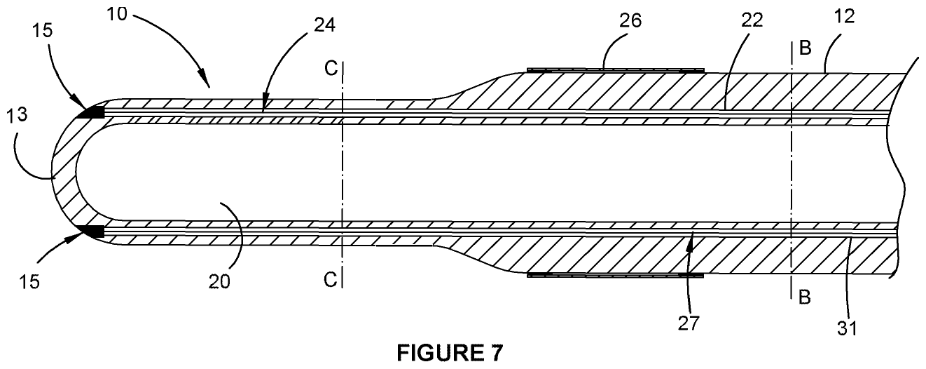

If the entire catheter is made of uniform material, differential tip deflection may also be achieved by narrowing of the walls of the catheter body 12 at the tip portion 16, as illustrated in FIG. 7. And in fact, unless the wall thickness of the required deflectable portion of the catheter is less than the rest of the catheter shaft, the whole catheter will bend into a single arc on the application of traction to the steering member. Curvature of the whole length of the catheter shaft will actually make insertion of the catheter more difficult. The degree of reduction in wall thickness and the length of the thinner portion of the tip of the catheter need to be custom designed to result in the optimal shape of the catheter tip to conform to the shape of the curved part of the urethra when traction is applied to the steering member. Advantageously, by reducing the wall thickness of the catheter body 12, the tip portion 16 becomes softer, which can also lead to a lower risk of damage to the urethra by penetration of the wall.

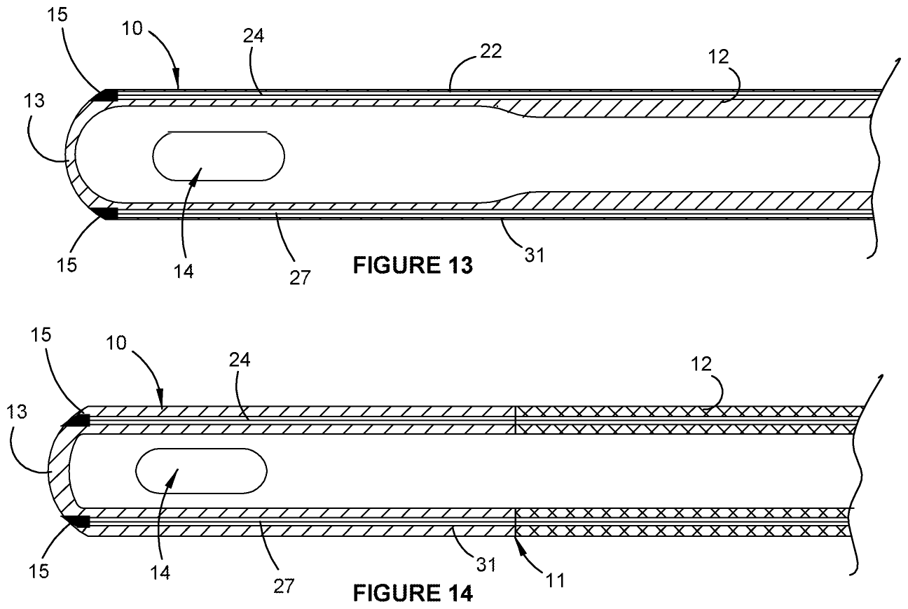

In a further embodiment of the catheter, the wall thickness of all or part of the tip portion of the catheter may be thinner than the elongate portion of the catheter, but the total outside diameter of the whole catheter may be constant, if the diameter of the catheter main channel 20 is greater in the tip portion than in the elongate portion of the catheter, as is shown in FIG. 13.

In other embodiments, the catheter body 12 may be formed with deformation lines, which may be linear, curved or radial or combinations thereof for example, to facilitate bending of the catheter body at the junction of the tip portion 16 and the catheter body 12. Those skilled in the art will appreciate that other means to induce bending, buckling, twisting or otherwise contorting of the catheter body 12 are also possible.

As will be described in more detail below, the tip portion 16 is configured for deflection in a plane coincident with a longitudinal axis of the elongate portion. This single plane deflection without any lateral or sideways movement is provided to allow the catheter 10 to take the shape of the urethra to facilitate insertion therein.

Deflection of the tip portion 16 is possible within an angle range of 0 to 90 degrees, to enable the catheter body 12 to follow the natural contour of the patient's urethra.

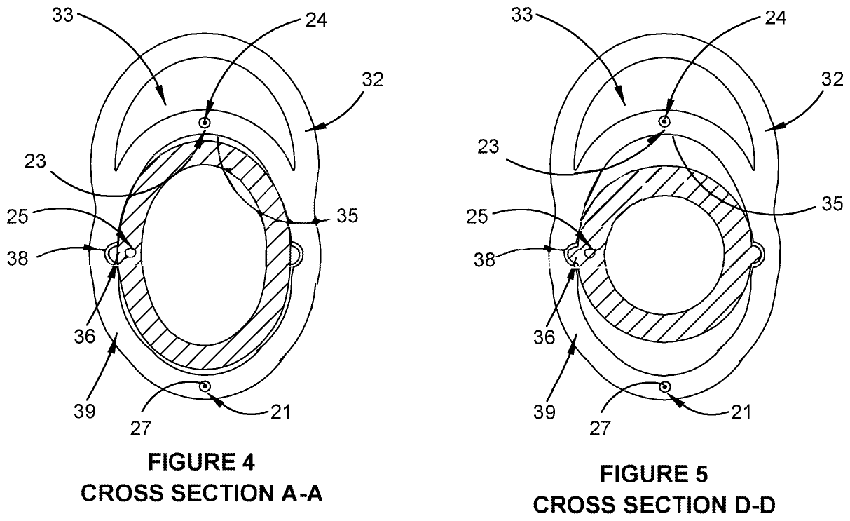

Catheter 10 also has a balloon channel 25 (FIGS. 4, 5, 8 and 12b) within the catheter body 12, in which fluid, e.g. water, can be communicated from a connector 28 disposed near a proximal end 40 of the catheter body 12 to an inflatable balloon 26 that, when inflated, helps retain the catheter 10 in position to allow drainage of the patient's bladder on an ongoing basis, typically over a number of days. The inflatable balloon 26 is spaced from the tip portion 16 and preferably sited proximal to or within the deflecting portion of the catheter 10. To maintain the balloon 26 in an inflated state while desired, valve 30 is provided. The above described components are typical of the commonly used Foley urethral catheter. Although illustrated with a single balloon 26, it will be appreciated that multiple balloons may also be used.

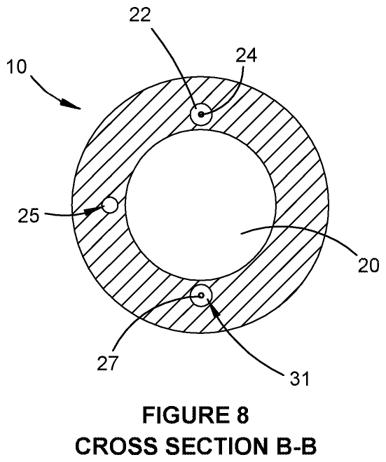

As illustrated in FIG. 8, the catheter body 12 has first and second further channels 22 and 31, each with a respective steering member 24 and 27 received therein. The first and second further channels 22 and 31 are positioned mid dorsally and mid ventrally, respectively, so that traction on the dorsal steering member 24 will result in upward deflection of the catheter tip portion, and traction on the ventral steering member 27 will result in downwards deflection of the catheter tip portion.

In preferred embodiments, the steering member is a flexible nylon string, though it will be appreciated that the steering member may be formed of other materials, particularly other polymers, and take other constructions. It may also be made of a metallic component.

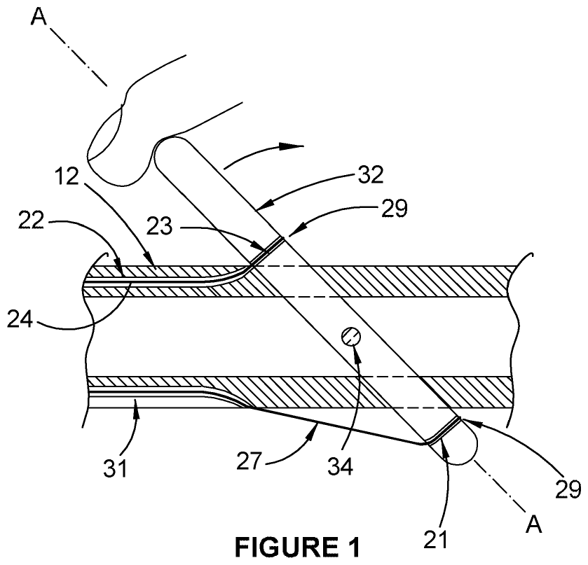

To enable a user to engage the steering members 24 and 27 to deflect the tip portion 16 of the catheter 10, the catheter 10 also includes an engagement member 32 that can be operated by the user. In one embodiment, such as that shown in FIGS. 1-5, the engagement member 32 is a lever.

The engagement member 32 is biased into an upright position to facilitate operation by a user. In this regard, by biasing the engagement member 32 into an upright position, a user can easily engage and operate member 32 with a single digit while the rest of the hand is used to hold the catheter 10 during introduction. FIGS. 1-3 show the engagement member 32 being engaged and moved to manipulate the tip portion 16. In use, a user will support the catheter body 12 at the engagement portion 18 with one hand and have their second hand further towards the distal end 13 to try to manipulate the catheter 10, and hold the patient's penis. As such, limited dexterity remains so it is important that the engagement member 32 be as accessible as possible.

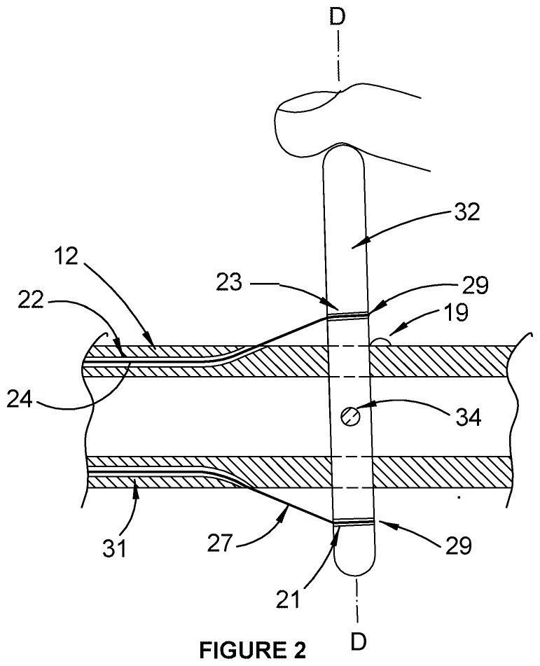

In some embodiments, the engagement member 32 may be biased into the upright position by tension on the steering member 24. Such a configuration is shown in FIG. 1. The pre-determined angle at which the engagement member 32 sits, in the undeflected state, may be determined by contact of the engagement member with the round catheter body, as in FIG. 1, by contact of the engagement member with mouldings of the catheter wall, as in FIG. 15, or contact with an engagement member limiter knob, as in FIG. 2.

In one embodiment, the maximum arc of rotation of the engagement member is determined and limited by a small knob 19, which is moulded onto the external wall of the catheter body 10 and may be situated at any appropriate point around the circumference of the external catheter wall. In FIG. 2, such an engagement member limiter 19 is sited dorsally, and will allow the engagement member to be rotated in an arc only up to 90 degrees to the horizontal. Such an engagement member limiter knob, may be manufactured such that the height of the knob will allow the engagement member to be pulled past the knob, with some resistance, but will then allow traction on the engagement member to be released, and for the engagement member to be ‘locked’ in the deflected state. Straightening of the catheter would then require forward pulsion of the engagement member to overcome the resistance offered by the engagement limiter knob.

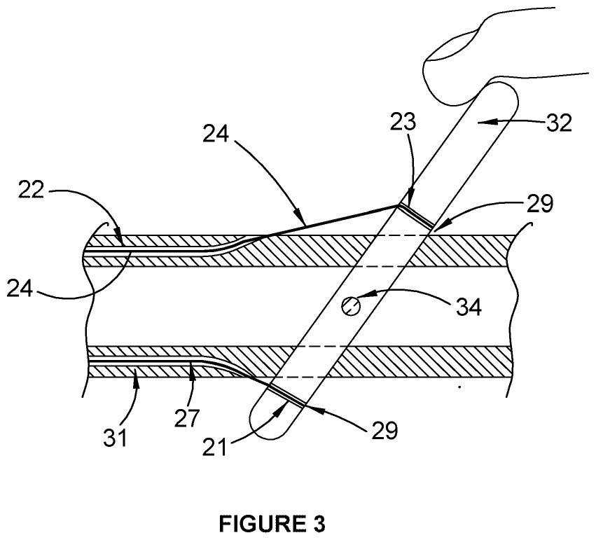

In other embodiments, the rotation of arc of the engagement member may be configured to be more than 90 degrees, as shown in FIG. 3. As above, limitation of rotation may be secondary to contact of the engagement member with the catheter body, the presence of an engagement member limiter or a moulding of the catheter body. It is also likely that limitation of rotation may be contributed to by the resilience of the catheter tip during deflection.

Markings on the catheter body 12 may be provided to allow a user to estimate the angle of deflection of the tip portion 16.

In the catheter described in international patent application no. PCT/AU2020/050972, the engagement member 32 consists of an inverted ‘U’ shaped lever, sitting above the catheter and in contact with the catheter body at bilateral socket joints, acting as a fulcrum for rotation of the engagement member as a lever. The currently described catheter, which is a variation of that catheter, incorporates an engagement member which is a full ring around the catheter body, rather than just a ‘half ring’. See FIGS. 4 and 5.

In this variation, the dorsal half of the ring forming the engagement member may be similar or identical to the dorsal component of the previously described catheter in PCT/AU2020/050972. The ventral component 39 may differ however from the dorsal component, as it does not need to be manipulated manually, and therefore does not need to protrude as far from the catheter body. Incorporation of a ventral ring component 39 does allow for the inclusion of a ventral steering member 27 within the catheter and thus the ability to actively deflect the catheter tip downwards, in addition to the active deflection upwards provided by the dorsal steering member 24.

Because of the previously described upward curvature of the male urethra, downward deflection of a catheter below the horizontal is not usually required to facilitate the catheter shape conforming to that of the urethra, during insertion of the catheter. (In these descriptions, the horizontal refers to a line projected proximally as a continuation of the line of the distal, straight, penile section of the male urethra.) If, however, the catheter is in the process of being inserted, and is already partly deflected above the horizontal, there may be a potential advantage in being able to actively deflect the catheter tip back down towards the horizontal, particularly if the urethral lining is irregular or not smooth. Active downward deflection may be advantageous in addition to the passive downward deflection which may occur due to the inherent resilience of the catheter.

In other embodiments, the engagement member 32 is shaped to sit on the catheter body 12 in an upright position. In the embodiments of FIGS. 4 and 5, the engagement member 32 has a curved or concave base that may abut the exterior surface of the catheter body 12, at one point of its arc of pivot.

As can be seen in FIGS. 4 and 5, the dorsal component of the engagement member 32 has a rounded base 35 that has a shape which is complementary with the outer surface of the hollow catheter 12 so that the engagement member 32 may sit on the hollow catheter 12. The rounded base 35 is rounded between lateral sides, as can be seen in FIGS. 4 and 5, but may be generally flat in its depth dimension (as viewed in FIG. 1) so as to sit on the catheter 12 in a generally stable manner so as to be presented to a user for easy engagement in use.

In the embodiment shown in FIG. 1, the rounded base 35 and tension of the steering member 24 may be sufficient to retain the engagement member 32 in a generally upright condition. In the embodiment shown in FIGS. 4 and 5, the engagement member 32 extends around the full circumference of the catheter body 12, and incorporates a socket on each side at the mid-point of the ring laterally, about which the engagement member is pivotable. The ventral component 39 of the engagement member 32 incorporates a channel 21, through which the ventral steering member 27 may pass, and then be fixed to the engagement member at the fixation point 29.

It will be appreciated that for the engagement member 32 to sit flush on the round catheter body 12 when angulated forward to the vertical as in FIG. 1, or backwards as in FIG. 3, the shape of the ring will in fact have to be oval rather than circular. Thus when the engagement member is located at any other point in its arc of rotation between these 2 extremes, the distance between the base of the engagement member 35 and the body of the catheter 12 will be greater than at those 2 extremes, and a maximum when the engagement member is aligned in the vertical upright position as in FIG. 5. Note that although the catheter shape appears to be oval in cross section A-A in FIG. 4, the catheter is in fact round, but appears oval only because the cross section A-A of the catheter is at an oblique angle as shown in FIG. 1.

In the embodiment of FIGS. 1, 2 and 3, the engagement member 32 acts as a lever coupled to the exterior of the catheter body 12 at point 34. This provides mechanical advantage to assist with deflection of the tip portion 16. As can be seen in FIGS. 1, 2 and 3, a knob 36 is formed on the sides of the catheter body 12 and corresponding sockets 38 are formed in the engagement member 32. the knobs 36 and sockets 38 cooperate to pivotably support the engagement member 32 to allow operation of the engagement member. It will be appreciated that in other embodiments the knobs 36 may be formed on the engagement member 32 and the sockets 38 formed in the catheter body 12. Again, it may be the base 35 of the engagement member 32 or the tension of the steering member 24 (or both) that keeps the engagement member 32 in position and upwardly biased for easy access by a user.

Because steering members 24 dorsally and 27 ventrally are configured to be symmetrical in their relationship to the catheter body, traction on one steering member will result in symmetrical and equal relaxation of traction on the opposite steering member, and thus allow for the engagement member to be controllable in 2 directions but in a single plane. Consequent on this control of forward and backward movement of the engagement member, the user will have control of active deflection both upwards and downwards of the catheter tip portion.

During manufacture of the engagement member 32, 2 holes may be incorporated into it, for passage of each steering member 24 and 27. During assembly, the engagement member 32 can then be passed over the catheter tip portion and engagement portion, and ‘clipped’ on to the catheter body 12, and the 2 steering members 24 and 27 pulled through holes 23 and 21 respectively in the engagement member 32, and secured using conventional securement means.

Aperture 33 is configured so that a user may insert a digit through the aperture for activation of the engagement member 32. The engagement member 32 and aperture 33 may take other forms or shapes, such as elliptical or complex shapes, such as an inverted ‘trigger’ and may be a simple lever without an aperture 33.

The above described engagement members 32 provide a simple, reliable and lightweight means for applying tension or relaxation of tension to steering members 24 and 27, to actively deflect the tip portion 16. This is important as urethral catheters are often retained in the urethra for many days at a time after insertion. Because patients potentially will have to walk and perform routine daily routines with a catheter indwelling, a deflection mechanism which is heavy or bulky will be completely unsuitable for purpose. As such, the bigger, heavier and more complex steering systems of catheters used to address the issue of curved anatomy in other medical fields are simply unsuitable for use with urethral catheters.

The above described catheter 10 is configured for use in catheterising the bladder of a male patient. In use it is inserted into the urethra of the patient and the tip portion 16 is then manipulated to facilitate insertion into the bladder. This may be by steering the catheter around curves of the urethra by deflecting the tip portion as the catheter is inserted into the urethra. Manipulation of the tip portion 16 may also be simply in order to dislodge the catheter from an otherwise impacted or jammed position and allow insertion to proceed.

The catheterisation process using catheter 10 is now described in more detail. The urethral anatomy and catheterisation process are shown in detail in diagrams in application PCT/AU2020/050972.

Immediately prior to insertion of the tip of the catheter into the urethra, the user places manual traction on the penis in order to steady it, and importantly to straighten the penile urethra, so that the catheter tip 13 is more likely to pass this part of the urethra without any resistance.

The catheter 10 is introduced with lubricant into the penile (distal) urethra and passed along the straightened distal urethra. This is usually the easiest part of insertion of a catheter, provided that the catheter 10 is straight and the urethra tube is anatomically normal.

Because the catheter is in its generally straight, undeflected form when it is introduced into the distal urethra, it usually passes easily through the generally straight penile urethra. However, the lining (mucosa) of the urethra can in fact be quite irregular, particularly if there has been any previous trauma, infection, instrumentation or operation. Irregularity of the urethra is particularly common after previous prostate surgery, especially TURP procedures.

As the catheter is gently pushed forward, if the tip impinges on any obstruction, due to any irregularity or defect of the urethral mucosa, the tip of the catheter can be slightly deflected upwards by the user, and then gently pushed further forward. This upward deflection of the catheter tip lifts it away from the posterior wall of the urethra. If the tip can't be advanced any further, then the catheter is gently withdrawn perhaps just a few millimetres, and the catheter tip is deflected just a little more. If the catheter then advances, no further deflection is applied, and the catheter is pushed through until urine flows out of the main catheter lumen.

With reference to the term ‘upwards’, those skilled in the art will appreciate that catheterisation is generally performed with a patient lying on their back and that an upwards direction would be towards a ceiling, though catheterisation may also be performed with a patient lying on their side or prone, in which case the deflection would be in a direction generally forward of their body.

As the catheter is pushed further forward after bypassing an obstruction, the traction on the steering member can be retained, resulting in persistent deflection of the catheter tip, or the traction can be released, allowing the catheter tip to passively straighten at least fractionally, prior to further advancement. Relaxing traction on the steering element after passing an obstruction may lower the risk that the deflected catheter tip may impinge on the anterior wall of the more proximal urethra. If active deflection upwards or passive straightening of the catheter tip do not allow free passage of the catheter tip up into the bladder, active pulsion forward of the engagement member can if necessary be applied by the single digit already in contact with the engagement member. This action will produce active downward deflection of the catheter tip back towards the horizontal.

The whole insertion process is performed using the manual sensation of resistance alone, and unlike other actively steerable devices, does not rely on either visual or radiological guidance. If the catheter readily passes without any resistance at all, it may not be necessary to actively deflect the catheter tip at all.

As the catheter tip reaches the curved junction of the bulbar urethra and the membranous urethra, difficulty may be encountered as the generally straight catheter meets a curve in the passage. Upon a user receiving tactile feedback by way of resistance that the tip is in this position, the catheter 10 may be retracted slightly (possibly only a few millimetres) and the engagement member 32 engaged to deflect the tip as the catheter 10 is subsequently advanced, thereby allowing the catheter 10 to pass through the curved junction of the bulbar urethral and the membranous urethra without impingement. This avoids the significant urethral trauma that can occur here, particularly in the form of false passages.

An alternative method of catheter introduction is characterised by continuous active deflection of the catheter tip, once active deflection has commenced, either by maintaining continuous, constant traction on the engagement member at the desired angle of deflection, or by ‘locking’ the engagement member by pulling it past the engagement member limiter, and then relaxing the traction on the engagement member until it rests against and behind the limiter.

Those skilled in the art will appreciate that the insertion process described is much simpler and less prone to local trauma than that currently generally utilised without the extra control provided by an actively steerable urethral catheter. This will particularly be the case where a patient has had a previously excavated prostate gland due to previous prostatic surgery such as a TURP procedure.

Owing to the above described configuration of the catheter, catheterisation can be performed solely using tactile feedback from the catheter.

Utilising anatomical knowledge of the male urethra has enabled the design of a new steerable urethral catheter which makes it specifically suitable for catheterisation of the male urethra by anyone who is physically able to pass a urethral catheter, including the patient himself. The steering control mechanism in preferred embodiments is a simple, single component, trigger-like lever, which requires a short, single digit movement. This single component can be manufactured very cheaply of plastic and attached to simple monofilament nylon strings.

Many previously designed steerable devices have required the strength of metal wire steering elements, some of which have been multi-filament to permit flexibility. This new design allows a nylon string to be used as the steering member because its larger size does not require the strength of steel, and full deflection can be achieved even with some elasticity of the steering element.

The advantages of a nylon steering element in a disposable catheter which may need to be retained in the bladder for several months include: cheapness and simplicity of manufacture; easier attachment to the catheter tip by heat fusion, a plastic plug or glue; potentially better glide characteristics in a silicone channel; absence of corrosion when in prolonged contact with fluids; absence of potential allergy to metallic components; easy sterilisation with the catheter without modification of the sterilisation technique; resistance to biofilm and potential infection, especially compared with a multi-filament wire; absence of radiological reflection interfering with the image on ultrasound, CT scan or MRI; and contra-indication of a metal component in an MRI scan, necessitating possible catheter removal prior to MRI.

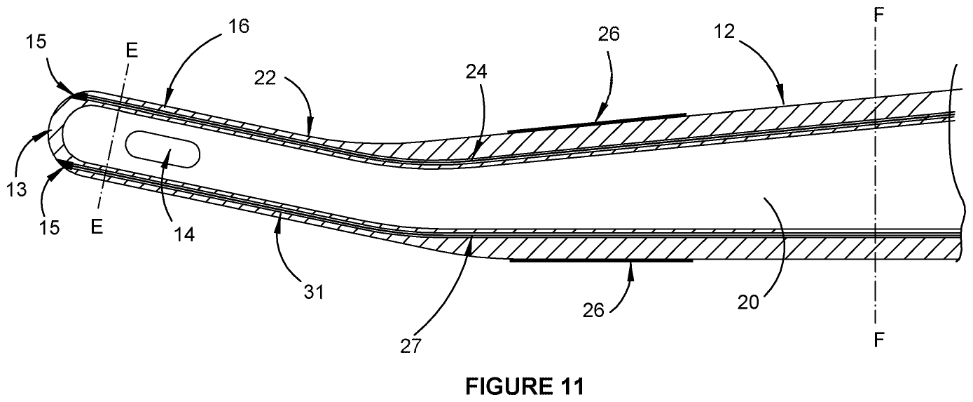

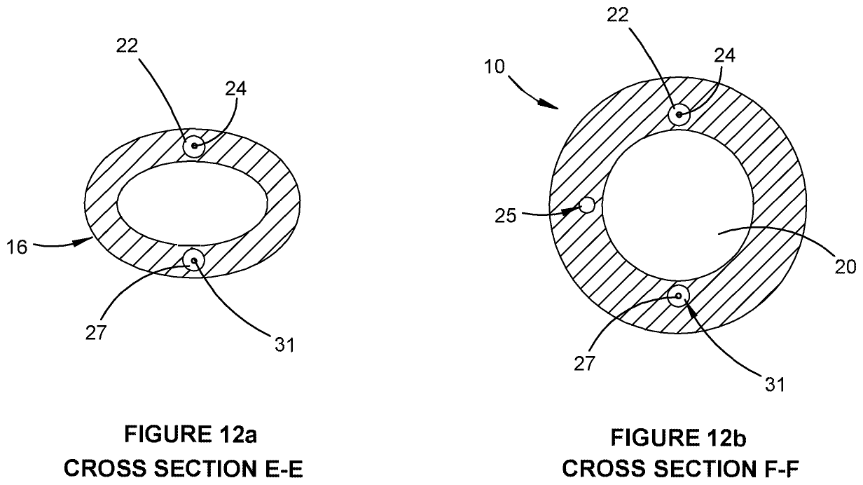

Many modifications of the above embodiments will be apparent to those skilled in the art without departing from the scope of the present invention. Catheter 12 may be other than round, such as elliptical for example, and may not have a consistent cross-sectional shape along its length. For example, the tip portion may have a flattened oval shaped cross-section, as in FIG. 12a, whereas the elongate portion of the same catheter may have a circular cross-section, as in FIG. 12b. These 2 figures represent cross sections E-E and F-F respectively, in FIG. 11. In this example, the thickness of the catheter wall is uniform around the circumference of the catheter within a single cross-section, although thicker in the elongate portion cross-section than in the tip portion cross-section.

In another embodiment, the tip portion of the catheter may have a circular lumen 20, but the lateral wall thickness of the catheter may be greater than the superior and inferior wall thickness of the catheter, as shown in FIG. 6, representing cross-section C-C of FIG. 7. This catheter wall shape is configured to inhibit lateral rotation of the catheter tip during active deflection. The elongate portion of the catheter may be circular, as is shown in FIG. 8, which represents the cross-section B-B of the catheter shown in FIG. 7. The elongate portion of the catheter may also be flattened in the antero-postero axis, in a similar shape to the tip portion shown in FIG. 6.

Many variations are possible of this basic concept of a non-circular catheter cross-section to promote deflection only in the plane coincident with the longitudinal axis of the elongate portion of the catheter, including in catheters with no active deflection, for which lateral rotation is also undesirable.

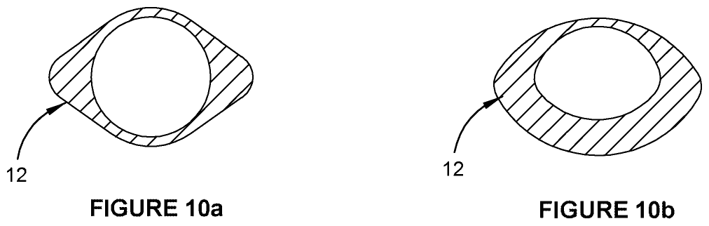

A cross-section of an example of a non-actively deflectable catheter is shown in FIG. 10a, in which the greater thickness of the lateral walls of the catheter, as opposed to the anterior and posterior walls, inhibits lateral rotation during insertion of a normal Foley catheter. Any lateral rotation might interfere with passive deflection of the catheter to conform to the shape of a normal urethra.

A cross-section of another example of a non-actively deflectable catheter tip portion is shown in FIG. 10b, in which lateral deflection is also inhibited by the increased thickness of the lateral walls of the catheter tip portion. In this example, the anterior wall thickness of the catheter is less than the posterior wall thickness, which should promote passive catheter deflection in the upward direction as opposed to the downward direction.

As an example of one more variation, the tip portion may be manufactured with a slight, fixed upward angulation as in FIG. 11, however unlike the upward angulation inherent in a traditional Coude tip catheter of up to 20 to 40 degrees, the angulation in the catheter described, is perhaps of the order of 10-20 degrees. Because of this lower angulation, the tip of the catheter can still fall within the projected line of the outside of the dorsal wall of the elongate portion of the catheter. There is therefore less risk of the catheter tip impinging on the urethral mucosa during passage up the straight section of the distal urethra, but still some potential advantage in the angulation promoting upward deflection of the catheter tip when it impinges on the curve of the bulbar urethra.

In international patent application PCT/AU2020/050972, in one embodiment of the catheter, there are 2 steering elements dorsally. It will be appreciated that in this extension of that patent application, it will be possible to have a combination of single or multiple steering elements, for example, there could be 2 steering elements dorsally and only one steering element ventrally.

Throughout this specification and the claims that follow, unless the context requires otherwise, the word “comprise”, and the variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### REFERENCE NUMERALS

10. Urinary catheter
11. Junction between catheter components of different composition
12. Hollow catheter body
13. Distal end
14. Drainage aperture
15. Plug
16. Tip portion
18. Engagement portion
19. Engagement member limiter
20. Main channel
21. Channel in ventral engagement member
22. Further channel
23. Channel in dorsal engagement member
24. Steering member
25. Balloon channel
26. Inflatable balloon
27. Steering member (ventral component)
28. Connector
29. Fixation point for steering member
30. Valve
31. Further channel (ventral)
32. Engagement member
33. Aperture
34. Pivot point
35. Round base of engagement member
36. Knob
37. Legs of engagement member
38. Socket
39. Ventral component of engagement member
40. Proximal end of catheter body

1. A urinary catheter, comprising a hollow catheter body, configured to be inserted within the urethra of a patient, the catheter body having an elongate portion, a tip portion and an engagement portion spaced from the distal end where a user can grasp the catheter, the tip portion being deflectable for the purpose of steering the catheter within the urethra to allow the catheter to follow a natural curvature of the urethra, the tip portion being movable from a generally straight condition to a deflected condition wherein deflection of the tip portion occurs while the elongate portion remains generally straight, wherein the catheter body has a main channel formed therein for draining urine from a bladder and at least 2 further channels formed remote from the main channel and in which flexible steering members can be received, the lumen of each further channel extending from the distal end to a location near the engagement portion with the steering members being secured within the further channels at the tip portion and configured for engagement by a user externally of the catheter to deflect the tip portion.

2. A urinary catheter according to claim 1, wherein the engagement member is attached to a dorsal steering member, which facilitates active upward deflection of the catheter tip portion.

3. A urinary catheter according to claim 1, wherein the engagement member has a ventral extension which allows for incorporation of a ventral steering member.

4. A urinary catheter according to claim 1, wherein the ventral steering member facilitates active downward deflection of the catheter tip portion.

5. A urinary catheter according to claim 1, wherein the tip portion may have an oval or flattened oval cross-sectional shape.

6. A urinary catheter according to claim 1, wherein the tip portion may have acutely angled lateral edges.

7. A urinary catheter according to claim 1, wherein the tip portion may be comprised of a different material to the elongate portion of the catheter, to facilitate differential deflectability of these two portions of the catheter.

8. A urinary catheter according to claim 1, wherein the tip portion may have thinner walls dorsally and ventrally, compared with the thickness of the lateral walls of the catheter.

9. A urinary catheter according to claim 1, wherein the dorsal wall thickness of the catheter tip portion may be thinner than the wall thickness of the ventral and lateral walls of the tip portion of the catheter.

10. (canceled)

11. A urinary catheter, according to claim 1, wherein the range of arc of rotation of the engagement member may be limited or locked by the incorporation of mouldings or a limiter knob or knobs on the external surface of the catheter wall.
